(19) United States
(12) Patent Application Publication
(10) Pub. No.: US 2008/0133146 A1
(43) Pub. Date: Jun. 5, 2008

(54) Device Having a Trend-Indicating Display

(76) Inventors: KC Shu Kun Chang, Evanston, IL (US); Jeffery S. Reynolds, Granger, IN (US); Benjamin Rush, Evanston, IL (US)

Correspondence Address: NIXON PEABODY LLP, 161 N. CLARK STREET, 48TH FLOOR, CHICAGO, IL, 60601, US

(21) Appl. No.: 11/793,117

(22) PCT Filed: Dec. 15, 2005
(86) PCT No.: PCT/US2005/045494

§ 371 Date: Jun. 14, 2007

Related U.S. Application Data
(60) Provisional application No. 60/637,242, filed on Dec. 17, 2004.

(51) Int. Cl. G01N 33/50

(52) U.S. Cl. 702/23

(57) ABSTRACT

A test device (10) for determining the analyte concentration in a current sample. A measuring unit (28) is included in the test device and is adapted to measure the reaction of a reagent and the analyte and to generate a signal indicative of the measured reaction. The test device (10) also includes a processor (32) that is electronically coupled to the measuring unit (28) and is adapted to determine the analyte concentration in the sample in response to receiving the signal indicative of the measured reaction from the measuring unit (28). Electronically coupled to the processor (32) is a memory (34), the memory (34) for storing the analyte concentration and including storage of a current sample and at least one past sample. The test device (10) further includes a user display (18) that is also electronically coupled to the processor (32), and a display automatically displaying the concentration of the current sample and at least one past sample in a graph.

### FIELD OF THE INVENTION

The present invention relates generally to liquid sample monitoring devices and, more particularly, to the manufacture and design of a display for use in a test device for determining the analyte concentration in a liquid sample.

### BACKGROUND OF THE INVENTION

Those who have irregular blood glucose concentration levels are often medically required to self-monitor their blood glucose concentration level. An irregular blood glucose level can be brought on by a variety of reasons, including illness, such as diabetes. The purpose of monitoring the blood glucose level is to determine the concentration level and then to take corrective action, based on whether the level is too high or too low, to bring the level back within a normal range. The failure to take corrective action can have serious medical implications.

Beyond the above-described blood glucose concentration level monitoring, self-testing systems are used for determining the presence or concentration of other analytes in body fluid, such as, for example, cholesterol, alcohol, and hemoglobin in blood or chemical substances in saliva. Beyond self-testing situations, portable test devices are also used to test for various types of chemicals in water and soil.

One method of monitoring a person's blood glucose level is with a portable, hand-held, blood glucose test device. A prior art blood glucose test device 6 is illustrated in FIG. 1. The portable nature of these devices 6 enables the users to conveniently test their blood glucose levels wherever the users may be. The test device 6 receives a test sensor 7 for harvesting the blood for analysis. The test sensor 7—one of which is required for each test—contains a reaction area including a reagent for producing a measurable reaction with the glucose indicative of the blood glucose concentration level. The test sensor harvests the blood, either prior or subsequent to insertion into the testing device, for reaction with the reagent stored within.

The device 6 contains a switch 8a to activate the device 6 and a display 9 to display the blood glucose analysis results. Alternatively, the device 6 is automatically activated upon receipt of the test sensor 7. To check the blood glucose level, a drop of blood is obtained from, for example, a lanced fingertip. The blood is harvested using the test sensor 7. The test sensor 7, which is inserted into a test device 6, is brought into contact with the blood drop. The test sensor 7 moves the blood to the inside thereof via, for example, capillary action. Alternatively, the blood sample is harvested with the test sensor 7 prior to inserting the test sensor 7 into the test device. The blood sample now within the test sensor 7 mixes with the reagent causing a reaction between the reagent and the glucose in the blood sample. The test device 6 then measures the reaction to determine the glucose concentration in the blood. Once the results of the test are displayed on the display 9 of the test device 6, the test sensor 7 is discarded. Each new test requires a new test sensor 7. There are different types of test sensors for use with different types of test devices. Electrochemical or optical (e.g., calorimetric) assays are two types of testing used to measure blood glucose concentration levels.

There is a need for an inexpensive test device that includes a user-friendly display for illustrating the user's past glucose readings.

### SUMMARY OF THE INVENTION

A test device for determining the analyte concentration in a current sample is disclosed according to one embodiment of the present invention. The test device has a measuring unit that is adapted to measure the reaction of a reagent and the analyte. A signal is generated that is indicative of the measured reaction. Electronically coupled to the measuring unit is a processor that is adapted to determine the analyte concentration in the sample in response to receiving the signal indicative of the measured reaction from the measuring unit. Also included in the test device is a memory electronically coupled to the processor. The memory stores the analyte concentration and includes storage of a current sample and at least one past sample. The memory may also store other pertinent information such as the time and date of the measurement as well as other notes (meal information, exercise information, control measurements, and other lifestyle information of interest in disease management). Also included in the test device is a user display that is electronically coupled to the processor. The user display automatically displays the concentration of the current sample and at least one past sample in a graph. The user display may also list the other pertinent information described above.

The above summary of the present invention is not intended to represent each embodiment or every aspect of the present invention. The detailed description and figures will describe many of the embodiments and aspects of the present invention.

### BRIEF DESCRIPTION OF THE DRAWINGS

The foregoing and other advantages of the invention will become apparent upon reading the following detailed description and upon reference to the drawings.

FIG. 1 is a top view of a prior art blood glucose test device.

FIG. 2 is a schematic of a glucose meter according to one embodiment of the present invention.

FIG. 3 is a functional block diagram of the test device of FIG. 2.

FIG. 4 is a functional block diagram of the test device of FIG. 2 according to an alternative embodiment of the present invention.

FIG. 5 is a view of one embodiment of a display to be used on the meter of FIG. 1.

FIG. 6 is a view of another embodiment of a display that can be used on the meter of FIG. 1.

FIG. 7 is view of a further embodiment of a display that can be used on the meter of FIG. 1.

FIG. 8 is a display screen with a line graph according to one embodiment.

### DESCRIPTION OF ILLUSTRATIVE EMBODIMENTS

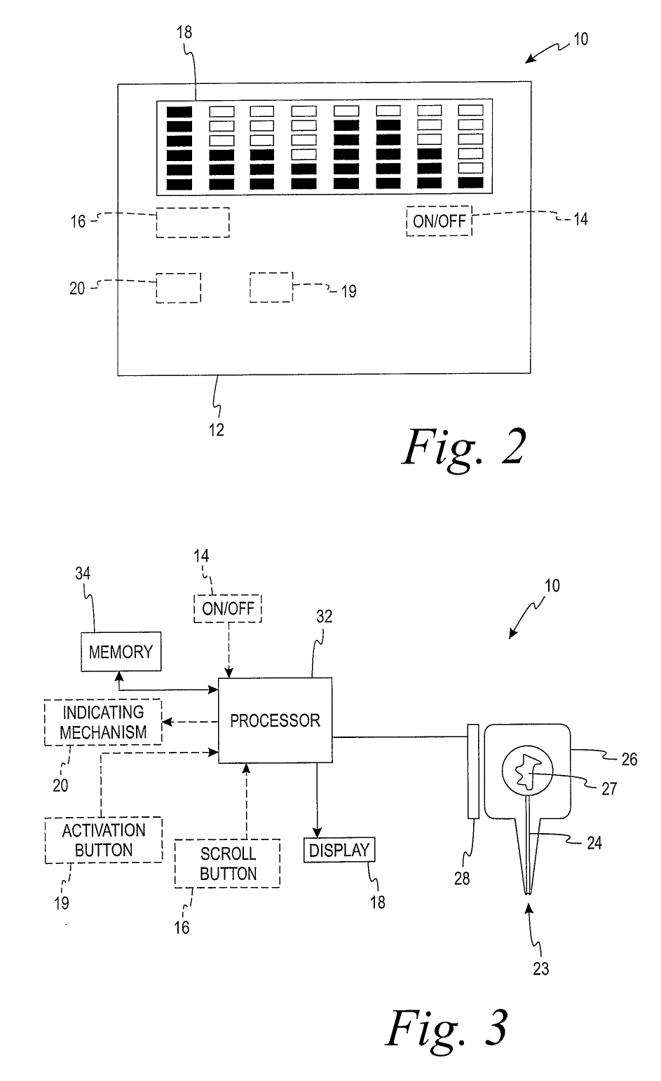

Referring now to FIG. 2, there is shown a test device 10 for determining a user's blood glucose concentration level according to one embodiment of the present invention. While the following discussion describes determining the glucose concentration in blood, it is understood that the present invention may be employed in determining the concentration of other analytes in other types of samples.

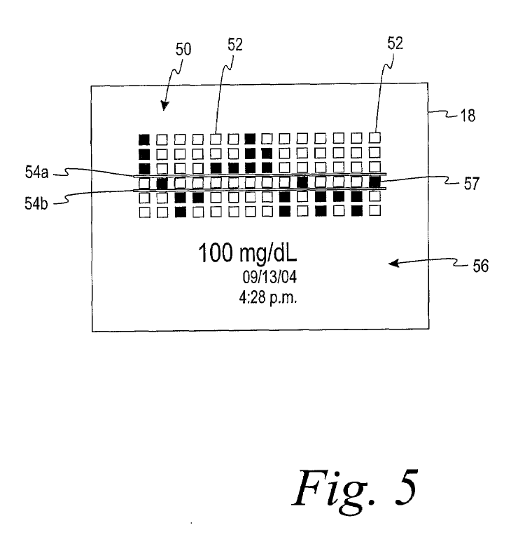

The test device 10 includes a housing 12, an optional power button 14, an optional scroll element or button 16, a display panel 18, an optional one-step activation button 19, and an optional indicating mechanism 20. The power button 14 is used to turn the test device 10 on and off. Alternatively, the test device 10 may automatically activate upon receipt of a test sensor. Alternatively, an initial activation (e.g., depression) of the scroll button 16 activates the test device 10. The display panel 18 displays the test results and will be described more fully with respect to FIGS. 5-7 below. The optional indicating mechanism 20 (e.g., an LED) is used to alert the user to an alarm condition, such as an abnormal reading, a glucose reading that is too high or too low, or another problem, with the test device 10. In an alternative embodiment, there is no indicating mechanism 20 and the display panel 18 is used to alert the user to the alarm condition. The test device 10 may also have an alphanumeric display 56 (FIG. 5) for displaying exact numeric readings and other information such as the time and date of the readings, the user's exercise and menu information, and other disease-control information.

Referring to FIG. 3, the internal components of the test device 10 will be described. The test device 10 includes a measuring unit 28 that receives a fluid collection apparatus or test sensor 26. In embodiments where calorimetric testing is implemented, the measuring unit comprises a spectrograph, a photometric measuring unit, or other optical measuring unit. The test sensor 26 includes a reagent 27 that reacts with a blood sample, creating a measurable reaction indicative of the concentration of glucose in the blood sample.

The type of reagent implemented in the test device 10 depends on the type of measuring used. For example, in calorimetric testing, the reagent reacts with the glucose in a blood sample causing a colorimetric reaction indicative of the glucose concentration level. A photometric measuring unit or other optical device reads the degree of color change. Colorimetric testing is described in detail in U.S. Pat. Nos. 6,181,417 (entitled “Photometric Readhead With Light Shaping Plate”), 5,518,689 (entitled “Diffuse Light Reflectance Readhead”), and 5,611,999 (entitled “Diffuse Light Reflectance Readhead”).

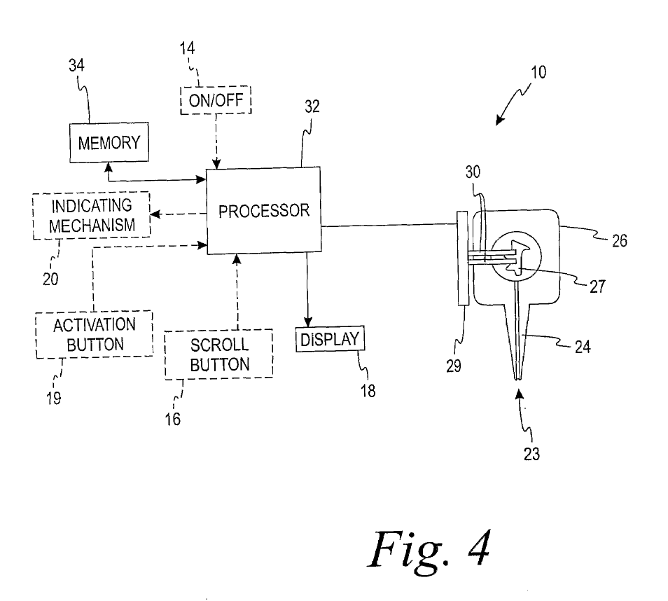

Referring also to FIG. 4, a test device 10 having an electrochemical measuring unit 29 is illustrated according to an alternative embodiment of the present invention. In an electrochemical assay, the reagent is designed to react with glucose in the blood to create an oxidation current at electrodes 30 that is directly proportional to the concentration of glucose in the user's blood. The current is measured by the electromechanical measuring unit 29, which is electrically coupled to the electrodes 30. An example of an electrochemical testing system is described in detail by commonly-owned U.S. Pat. No. 5,723,284 (entitled “Control Solution And Method For Testing The Performance Of An Electrochemical Device For Determining The Concentration Of An Analyte In Blood”).

Referring now to either FIG. 3 or 4, the test device 10 includes a processor 32 that is electrically coupled to the measuring unit 28 (FIG. 3) or the electromechanical measuring unit 29 (FIG. 4) and the power button 14. The processor 32 calculates the blood glucose level and outputs the result to the display 18. The processor 32 may also be connected to a memory 34 for storing information regarding past glucose readings, such as the blood glucose level, and the date and time of measurement. Alternatively, the processor 32 may store this information.

Turning now to FIG. 5, an embodiment of the display 18 will be described. In this embodiment, the display 18 includes a bar graph display 50 which is made up of a plurality of discrete sections or a plurality of boxes 52. For example, in a liquid crystal-type display, these discrete sections could be segments or pixels. The vertical axis of the bar graph display represents the approximate concentration of the glucose in the sample, while the horizontal axis represents the time that the sample was obtained. In this embodiment, the bar graph includes six boxes 52 arranged vertically to represent six different ranges of glucose readings. For example, each box may represent a range of approximately 75 mg/dL. In previous test devices with graphical displays, the graphical display plots the exact reading of the glucose concentration. This is often more information than the user needs and also requires a more costly display.

In this embodiment, the bar graph also includes two horizontal lines 54a, 54b. The two lines 54a, 54b are shown to clearly illustrate to the user a “normal” or average glucose concentration. The boxes above the line 54a indicate “high” glucose concentrations, while the boxes below the line 54b indicate “low” glucose readings. In some embodiments, the three different types of concentrations may be indicated by separate colors or another form of demarcation, or there may be no visual demarcation at all as to whether a concentration is “high,” “low,” or “normal.”

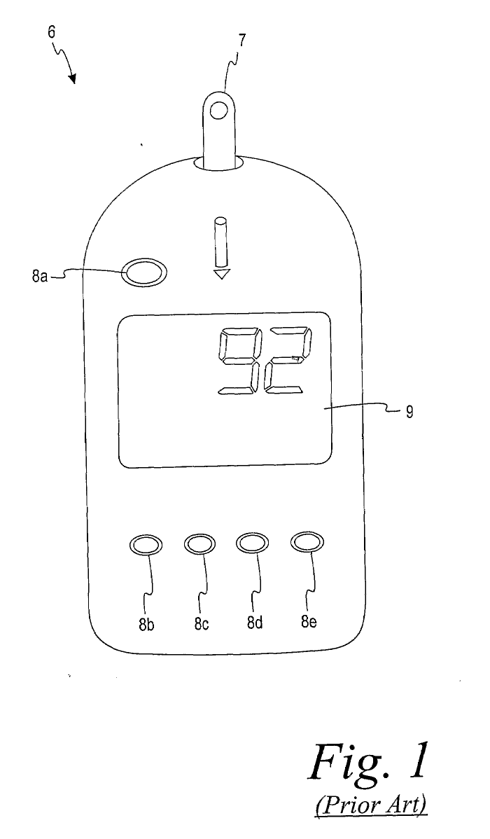

Below the bar graph, a numerical display 56 indicates the date, time, and exact concentration of a most current sample 57. Using the scroll button 16 (FIGS. 1-4), the user can scroll from the most recent sample through past samples. The data for the past samples may be stored in the memory 34 (FIG. 2) or in the processor 32 (FIG. 2). As the user scrolls, the display screen will highlight the various samples. Also, in some embodiments, the numerical display 56 will display the exact concentration level and the date and time when the highlighted sample was measured. In some embodiments, the display screen 50 may not include a numerical display, but only the graph of the concentrations.

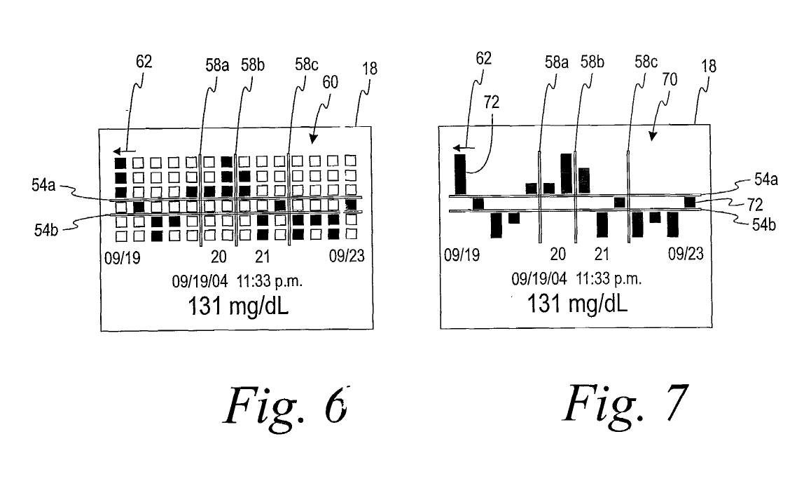

Turning now to FIG. 6, another embodiment of the display screen 18 is shown. In this embodiment, a bar graph 60 includes a plurality of vertical lines 58a, 58b, 58c that demarcate specific periods of time (e.g., a day). For example, in the embodiment shown in FIG. 6, the samples shown between lines 58a and 58b comprise all the samples taken on one day. This allows a user to quickly review how the concentration levels of the samples varied over a one day period, or if the user had an especially bad or good day.

In this embodiment, a scroll arrow 62 is also illustrated. The scroll arrow 62 indicates that the user can also view other readings. The arrow 62 can be on either side of the screen 62, depending on in which direction the graph extends.

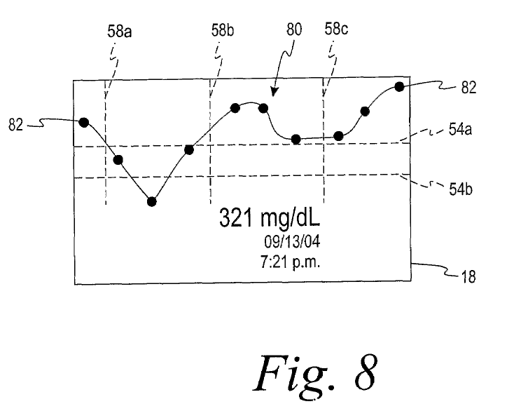

Another embodiment of the display screen 18 is shown in FIG. 7. In this embodiment, a bar graph 70 does not include the discrete boxes 52 shown in FIGS. 5 and 6. Instead, the samples consist of continuous bars 72. These bars provide the user with an indication of the amount of the glucose reading. The bars may be drawn to represent the exact concentration of the samples (i.e., a reading of 70 mg/dL will be lower than a reading of 75 mg/dL), or the bars may still represent ranges of concentrations. In FIG. 7, the graph 70 includes the plurality of vertical lines 58a, 58b, 58c demarcating periods of time and horizontal lines 54a, 54b separating the “normal” reading from the high and low readings. The test device 10 could be programmed to allow the user to select the demarcation values between the low, normal, and high ranges.

In some embodiments, the graph may be a line graph 80, such as the one shown in FIG. 8. FIG. 8 illustrates the display screen 18 with the line graph 80. Each glucose concentration sample is indicated by a point 82 on the graph 80. The graph 80 may or may not include the plurality of vertical lines 58a, 58b, 58c, or horizontal lines 54a, 54b.

In any of the embodiments described above, the display 18 may automatically and/or continuously display the user's current and past readings. Alternatively, the one-step activation system 19 may be included to allow the user to switch between a display of current and past readings and a screen with other information such as alerts. The one-step activation button 19 may be a toggle-button. Alternatively, activation of the scroll button 16 may activate the trend indicating display on the display 18.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention.

### Alternative Embodiment A

A test device for determining the concentration of an analyte in a current sample, the test device comprising:

a measuring unit adapted to measure the reaction of a reagent and the analyte and to generate a signal indicative of the measured reaction;

a processor electronically coupled to the measuring unit, the processor being adapted to determine the analyte concentration in the current sample in response to receiving the signal indicative of the measured reaction from the measuring unit;

a memory electronically coupled to the processor adapted to store the analyte concentration, the memory including storage of a current sample and at least one past sample;

and

a trend-indicating display electronically coupled to at least one of the processor and memory, the trend-indicating display adapted to display the approximate analyte concentration of the current sample and the at least one past sample.

### Alternative Embodiment B

The device of embodiment A further comprising, at most, a one-step activation system that triggers the display, the activation system adapted to cause the concentration of the current sample of the analyte and the concentration of at least one past sample to be displayed on the trend-indicating display.

### Alternative Embodiment C

The device of embodiment B wherein the at most one-step activation system is a single toggle button.

### Alternative Embodiment D

The device of embodiment A wherein the trend-indicating display displays the concentration of the current sample in numeric form.

### Alternative Embodiment E

The device of embodiment A wherein the trend-indicating display displays the concentration of the current sample in graph form.

### Alternative Embodiment F

The device of embodiment E wherein the trend-indicating display displays the graph is a bar graph.

### Alternative Embodiment G

The device of embodiment F wherein the bar graph includes a plurality of discrete sections, each of the plurality of discrete sections corresponding to a range of concentration levels.

### Alternative Embodiment H

The device of embodiment G wherein the bar graph is segregated into sections indicating high concentration levels, normal concentration levels, and low concentration levels.

### Alternative Embodiment I

The device of embodiment E wherein the graph is a line graph.

### Alternative Embodiment J

The device of embodiment I wherein the line graph is segregated into sections indicating high concentration levels, normal concentration levels, and low concentration levels.

### Alternative Embodiment K

The device of embodiment E wherein the graph is segregated vertically by time periods based on the time the current and at least one past sample was measured.

### Alternative Embodiment L

The device of embodiment K wherein the time period is a day.

### Alternative Embodiment M

The device of embodiment A wherein the test device includes a scroll button for allowing the user to move a cursor so as to highlight one of the current sample or the at least one past sample.

### Alternative Embodiment N

The device of embodiment M wherein the display provides numeric data regarding the highlighted sample.

### Alternative Embodiment O

The device of embodiment N wherein the numeric data includes a concentration level, and a date and time that the highlighted sample was measured.

### Alternative Embodiment P

The device of embodiment A wherein the reagent is adapted to produce an optical reaction and the measuring unit is adapted to measure the optical reaction.

### Alternative Embodiment P

The device of embodiment P wherein the optical reaction is a calorimetric reaction and the measuring unit is adapted to measure the calorimetric reaction.

### Alternative Embodiment R

The device of embodiment A wherein the reagent is adapted to produce an electrochemical reaction and the measuring unit is adapted to measure the electrochemical reaction.

### Alternative Embodiment S

The device of embodiment A wherein the sample is blood.

### Alternative Embodiment T

The device of embodiment A wherein the analyte is glucose.

### Alternative Embodiment U

The device of embodiment A wherein the trend-indicating display is a low-resolution display.

### Alternative Embodiment V

The device of embodiment A, wherein the trend-indicating display is a segmented display.

Alternative Process W

A method for displaying a plurality of samples on a test device, the test device having a memory in which a concentration of at least one past sample is stored, the test device being adapted to receive a test sensor for collecting the sample, the test sensor containing a reagent adapted to produce a reaction indicative of an analyte concentration in the sample, the method comprising the acts of:

measuring the reaction between an analyte in a current sample and the reagent contained in the test sensor;

determining the analyte concentration of the analyte in a body fluid; and

displaying the approximate concentration of the current sample of the analyte and the concentration of at least one past sample on a trend-indicating display.

Alternative Process X

The method of process W wherein the displaying comprises either continuously displaying or displaying in response to an activation of a one-step activation system.

Alternative Process Y

The method of process X wherein the one-step activation system is a single toggle button and the trend-indicating display is displayed after the single-toggle button is activated.

Alternative Process Z

The method of process W further comprising displaying the exact concentration of the current sample in numeric form.

Alternative Process AA

The method of process W wherein the displaying displays the concentration of the current sample in graph form.

Alternative Process AB

The method of process AA further comprising highlighting one of the current sample or the at least one past sample in the graph.

Alternative Process AC

The method of process AB further comprising displaying numeric data regarding the concentration of the highlighted sample.

Alternative Process AD

The method of process AC wherein displaying the numeric data includes displaying an exact concentration level, and a date and time that the highlighted sample was measured.

Alternative Process AE

The method of process AA wherein the graph is a bar graph.

Alternative Process AF

The method of process AE further comprising sectioning the bar graph into a plurality of discrete sections, each of the plurality of discrete sections corresponding to a range of approximate concentration levels.

Alternative Process AG

The method of process AF further comprising segregating the bar graph into sections indicating high concentration levels, normal concentration levels, and low concentration levels.

Alternative Process AH

The method of process AA wherein the graph is a line graph.

Alternative Process AI

The method of process AH further comprising segregating the line graph into sections indicating high concentration levels, normal concentration levels, and low concentration levels.

Alternative Process AJ

The method of process AA further comprising segregating the graph vertically by time periods based on the time the current and at least one past sample was measured.

Alternative Process AK

The method of process AJ wherein the time period is a day.

Alternative Process AL

The method of process W wherein measuring comprises measuring an optical reaction.

Alternative Process AM

The method of process W wherein measuring comprises measuring a calorimetric reaction.

Alternative Process AN

The method of process W wherein measuring comprises measuring an electrochemical reaction.

Alternative Process AO

The method of process W wherein the sample is blood.

Alternative Process AP

The method of process W wherein the analyte is glucose.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

1-42. (canceled)

43. A test device for determining the concentration of one or more analytes in a sample, the test device comprising:
a measuring unit for measuring a plurality of reactions between a reagent mixture and the one or more analytes, wherein the measuring unit generates a plurality of signals, each of the plurality of signals being indicative of a particular reaction;
a processor in communication with the measuring unit, wherein the processor processes each of the plurality of signals generated by the measuring unit so as to determine the concentration of the one or more analytes in the sample;
a memory in communication with the processor, wherein the memory stores the concentrations of the one or more analytes, the memory including storage of a current sample and at least one past sample;
a trend-indicating display that upon actuation displays a graphical representation illustrating a relationship between the current sample and the at least one past sample; and
a one-step activation system that triggers the display, the activation system adapted to cause the concentration of the current sample of the analyte and the concentration or trend of the at least one past sample to be displayed on the trend-indicating display.

44. The device of claim 43, wherein the one-step activation system is a single toggle button.

45. The device of claim 43, wherein the trend-indicating display displays the concentration of the current sample in numeric form.

46. The device of claim 43, wherein the trend-indicating display displays the concentration of the current sample in graph form.

47. The device of claim 46, wherein the trend-indicating display displays the graph as a bar graph.

48. The device of claim 47, wherein the bar graph includes a plurality of discrete sections, each of the plurality of discrete sections corresponding to a range of concentration levels.

49. The device of claim 48, wherein the bar graph is segregated into sections indicating high concentration levels, normal concentration levels, and low concentration levels.

50. The device of claim 46, wherein the graph is a line graph.

51. The device of claim 50, wherein the line graph is segregated into sections indicating high concentration levels, normal concentration levels, and low concentration levels.

52. The device of claim 46, wherein the graph is segregated vertically by time periods based on the time the current and at least one past sample was measured.

53. The device of claim 43, where the test device includes an interface for allowing a user to highlight one of the current sample or the at least one past sample.

54. The device of claim 53, wherein the interface includes a scroll button for allowing the user to move a cursor so as to highlight one of the current sample or the at least one past sample.

55. The device of claim 43, wherein the trend-indicating display is a segmented display.

56. A method for displaying a plurality of concentrations on a test device, the test device having a memory in which a concentration of at least one past sample is stored, the test device being adapted to receive a test sensor for collecting a sample, the test sensor containing a reagent adapted to produce a reaction indicative of the concentration of one or more analytes in the sample, the method comprising the acts of:
measuring the reaction between the one or more analytes in the sample and the reagent contained in the test sensor;
determining the concentration of the one or more analytes in a body fluid; and
displaying the concentration of the one or more analytes and at least one past analyte concentration on a trend-indicating display, the displaying comprising either continuously displaying or displaying in response to an activation of a one-step activation system.

57. The method of claim 56, wherein the one-step activation system is a single toggle button and the trend-indicating display is displayed after the single-toggle button is activated.

58. The method of claim 56, further comprising highlighting the concentration of the one or more analytes or the at least one past analyte concentration via a user interface.

59. The method of claim 56, wherein the displaying displays the concentration of the one or more analytes and the at least one past analyte concentration or trend in graph form.

60. The method of claim 59, wherein the graph is a bar graph.

61. The method of claim 60, further comprising sectioning the bar graph into a plurality of discrete sections, each of the plurality of discrete sections corresponding to a range of approximate concentration levels.

62. The method of claim 61, further comprising segregating the bar graph into sections indicating high concentration levels, normal concentration levels, and low concentration levels.

63. The method of claim 59, wherein the graph is a line graph.

64. The method of claim 63, further comprising segregating the line graph into sections indicating high concentration levels, normal concentration levels, and low concentration levels.

65. The method of claim 59, further comprising segregating the graph vertically by time periods based on the time the concentration of the one or more analytes and the at least one past analyte concentration were measured.

66. A test device for determining the concentration of one or more analytes in a sample, the test device comprising:
a measuring unit for measuring a plurality of reactions between a reagent mixture and the one or more analytes, wherein the measuring unit generates a plurality of signals, each of the plurality of signals being indicative of a particular reaction;
a processor in communication with the measuring unit, wherein the processor processes each of the plurality of signals generated by the measuring unit so as to determine the concentration of the one or more analytes in the sample;
a memory in communication with the processor, wherein the memory stores the concentrations of the one or more analytes, the memory including storage of a current sample and at least one past sample;
a trend-indicating display that upon actuation displays a graphical representation illustrating a relationship between the current sample and the at least one past sample; and
an interface for allowing a user to highlight one of the concentrations of the one or more analytes or the at least one past analyte concentration.

67. The device of claim 66, wherein the interface includes a scroll button for allowing the user to move a cursor so as to highlight one of the concentrations of the one or more analytes or the at least one past analyte concentration.

68. The device of claim 66, further comprising a one-step activation system that triggers the display, the activation system adapted to cause the concentration of the one or more analytes and the concentration or trend of the at least one past analyte concentration to be displayed on the trend-indicating display.

69. The device of claim 68, wherein the one-step activation system is a single toggle button.

70. A method for displaying a plurality of concentrations on a test device, the test device having a memory in which a concentration of at least one past sample is stored, the test device being adapted to receive a test sensor for collecting a sample, the test sensor containing a reagent adapted to produce a reaction indicative of the concentration of one or more analytes in the sample, the method comprising the acts of:
measuring the reaction between the one or more analytes in the sample and the reagent contained in the test sensor;
determining the concentration of the one or more analytes in a body fluid;
displaying the concentration of the one or more analytes and at least one past analyte concentration on a trend-indicating display; and
allowing a user to highlight the concentration of the one or more analytes or the at least one past analyte concentration on the trend-indicating display via a user interface.

71. The method of claim 70, wherein the interface includes a scroll button for allowing the user to move a cursor so as to highlight the concentration of the one or more analytes or the at least one past analyte concentration.

72. The method of claim 70, the displaying including either continuously displaying or displaying in response to an activation of a one-step activation system.

73. The method of claim 72, wherein the one-step activation system is a single toggle button and the trend-indicating display is displayed after the single-toggle button is activated.
